(19) United States
(12) Patent Application Publication
(10) Pub. No.: US 2015/0265685 A1
(43) Pub. Date: Sep. 24, 2015

(54) APYRASE THERAPY FOR FIBROPROLIFERATIVE DISORDERS, PULMONARY HYPERTENSION, AND HEART FAILURE

(71) Applicant: APT Therapeutics, Inc., St. Louis, MO (US)

(72) Inventors: Ridong Chen, Naperville, IL (US); Soon Seog Jeong, Naperville, IL (US)

(21) Appl. No.: 14/666,121

(22) Filed: Mar. 23, 2015

Related U.S. Application Data
(60) Provisional application No. 61/968,539, filed on Mar. 21, 2014.

(51) Int. Cl. A61K 38/46; A61K 45/06

(52) U.S. Cl. CPC A61K 38/46; A61K 45/06; C12Y 306/01005

(57) ABSTRACT

This invention provides new methods of treating subjects with fibroproliferative disease such as pulmonary hypertension (e.g. pulmonary arterial hypertensions), posthrombotic syndrome associated with venous thrombosis, or ventricular heart failure associated with fibroproliferative disease. In each case, the treatment methods of the present invention comprise administering apyrase agents—for example, a soluble agent belonging to the class of CD39 apyrases, optionally CD39L apyrase family, or optionally CD39L3 apyrases. Also provided are methods of diagnosing pulmonary arterial hypertension comprising quantifying cytokines in biological fluids and examining the values for those elevated in comparison to normal control values.

### CROSS REFERENCE TO RELATED APPLICATIONS

This application claims the benefit of U.S. Provisional Application No. 61/968,539 filed Mar. 21, 2014.

### REFERENCE TO A SEQUENCE LISTING

The instant application, contains a Sequence Listing which has been submitted via EFS-Web and is hereby incorporated by reference in its entirety.

### TECHNICAL FIELD

The present invention relates to apyrase agents, methods for treating subjects with fibroproliferative disease, pulmonary hypertension, heart disease, methods of diagnoses, and surrogate markers thereof.

### BACKGROUND

Fibrosis is the formation or development of excess connective tissue in an organ or tissue, which is characterized by accumulation of fibrotic material (e.g., extracellular matrix) following tissue damage. While this process can be essential to tissue and organ repair, in its maladaptive forms, it can result in pathologies of clinical importance. This maladaptive process, referred to here and elsewhere as fibroproliferative disease (or “PFD”), can lead to structural modifications of architecture of the organ or tissue involved and result in functional loss.

Fibroproliferative disease can affect almost any organ or tissue and is associated with a wide variety of diseases and injuries, especially in organs with frequent exposure to chemical and/or biological insults including lung, skin, digestive tract, kidney and liver. Fibroproliferative disease is responsible for morbidity and mortality associated with vascular diseases, such as cardiac disease, cerebral disease, and peripheral vascular disease, and with organ failure in a variety of chronic diseases affecting the pulmonary system (asthma, cystic fibrosis, IPF, COPD, pulmonary arterial hypertension, ARDS), renal system (diabetic nephropathy, lupus nephritis, FSGS, IgA nephropathy, transplant nephropathy), eyes (dry eye, diabetic macular edema, diabetic retinopathy, glaucoma, AMD), cardiac system (atherosclerosis, myocardial infarction, endomyocardial fibrosis, congestive heart failure), hepatic system (congenital hepatic fibrosis, alcoholic liver disease, HCV/HBV, primary sclerosing cholangitis, idiopathic portal HTN, nonalchoholic steatohepatitis, autoimmune hepatitis, primary biliary cirrhosis), digestive system, and skin (scleroderma, keloids, hypertrophic scars, eosinophilic fasciitis, dermatomyositis). Hence, fibrosis is a leading cause of morbidity and mortality. Nearly 50% of all deaths in the developed world are associated with some type of chronic fibroproliferative disease.

Fibroproliferative disease is apparent in Pulmonary hypertension (PH) type I (also known as pulmonary arterial hypertension or “PAH”), defined as a mean pulmonary arterial pressure greater than 25 mm Hg at rest or greater than 30 mm Hg during exercise. It is characterized by a progressive and sustained increase in pulmonary vascular resistance that can lead to right ventricular (RV) failure. The other four types of PH are venous, hypoxic, thromboembolic and miscellaneous PH.

PAH is characterized by elevated pulmonary vascular resistance that increases right ventricular (RV) workload, eventually leading to death due to RV failure. PAH mainly affects young and middle-aged women.

There are 10,000-20,000 US patients living with PAH, with many more who are undiagnosed. The average age of diagnosis is 36 years. Nine vasodilator therapies, classified as prostacyclin analogs, endothelin receptor antagonists, phosphodiesterase-5 inhibitors, and guanylyl cyclase stimulators (riociguat approved in 2013) have been approved for the improvement of exercise capacity for 3 to 4 months. Recently, Macitentan (dubbed Opsumit), a small molecule modified from Bosentan, has been shown to significantly reduce morbidity but did not reduce mortality significantly in a long-term trial of 2 years. A meta-analysis of randomized controlled trials in PAH reported only a modest 3 mmHg reduction in mean PAP after treatment.

Recently, a sobering report noted that the 3 year survival for PAH patients managed with state-of the-art multiple drug therapy was only 58%, marginally better than in 1980s when there was no treatment at all. Moreover, these drugs do not protect patients from developing severe pulmonary vascular disease. Meanwhile, the treatment is costly (e.g., $75,000/yr for epoprostenol, $89,038/yr for Treprostinil, and $36,208/yr for Bosentan). When medical treatment fails, the final therapeutic option is lung and/or heart-lung transplantation.

Chronic pulmonary vasoconstriction appears to play only a minor role in severe PAH. The major factor responsible for the high pulmonary vascular resistance in severe PAH is FPD, wherein there is formation of occlusive neointimal and plexiform lesions in small, peripheral pulmonary arteries (Voelkel N F et al. Pathobiology of pulmonary arterial hypertension and right ventricular failure. Eur Respir J. 40: 1555-1565, 2012). Importantly, the survival of PAH patients was not determined by a vasodilator-induced decrease of pulmonary resistance, but rather by an improved RVEF (Boggaard H J et al. Chronic pulmonary artery pressure elevation is insufficient to explain right heart failure. Circulation. 120: 1951-1960, 2009). Elevated PAP, myocardial fibrosis and decreased capillary density collectively contribute to the RV failure (Drake J I et al. Molecular signature of a right heart failure program in chronic severe pulmonary hypertension. Am J Respir Cell Mol Biol. 45: 1239-1247, 2011; Voelkel N F et al. Mechanisms of right heart failure-A work in progress and a plea for failure prevention. Pulm Circ. 3: 137-143, 2013; Tamodiuniene R, Nicolls M R. Regulatory T cells and pulmonary hypertension. TCM 21: 166-171. 2011). RV failure is reversible, as the end-stage RV failure of PAH patients fully recovers within few weeks after lung transplantation. It is noteworthy that Sildenafil, the FDA-approved phosphodiesterase 5A inhibitor, increased RV fibrosis by 4 times in experimental PAH, an effect that may worsen the RV outcome.

In addition, autoimmune diseases, including systemic sclerosis, systemic lupus erythematosus, Hashimoto's thyroiditis, Sjogren's syndrome and the anti-phospholipid antibody syndromes, are associated with certain forms of PAH. Preclinical studies of athymic rats treated with SU5416 and adoptive T cell transfer show that abnormal regulatory T (T_{reg}) cell activity and immune dysregulation are predisposed to developing PAH following vascular injury (Taraseviciene-Stewart L et al. Inhibition of the VEGF receptor 2 combined with chronic hypoxia causes cell death-dependent pulmonary endothelial cell proliferation and severe pulmonary hypertension. FASEB J. 15: 427-438, 2001).

Recently, therapeutic approaches targeted to vascular remodeling and proliferation and RV function have been evaluated. Imatinib (Gleevec) is an antiproliferative agent developed to target the BCR-ABL tyrosine kinase in patients with chronic myeloid leukemia. In addition, the inhibitory effects of imatinib on PDGF receptor α and β and c-KIT inhibited mitogenic processes and suggested that it may be efficacious in PAH. As an add-on therapy in the disappointing Phase III trial, Imatinib improved exercise capacity and hemodynamics in PAH patients, but caused unacceptable pulmonary and cardiac toxicity with adverse events in 53% of patients. β-adrenergic receptor blockade such as carvedilol is cardioprotective and reduces mortality by about 30% in patients with LV heart failure, but is not used routinely in PAH. Recent studies in a rat SU+HX model of PAH demonstrated that treatment with carvedilol resulted in increased exercise endurance, improved cardiac output, and reduced fibrosis and rarefaction, and exercise-induced mortality. Nevertheless, the treatment did not improve mPAP or RV systolic pressure. There was a concern about the detrimental effects on hemodynamics and exercise capacity. Overall, each of current methods, suffers from one or multiple drawbacks such as lack of effectiveness, serious side effects, low patient compliance, and high cost.

Current treatments of PFD target the inflammation response which is believed to play a role in the development of fibrosis generally. Examples of pharmaceutical strategies for treating fibrosis include the use of immunosuppressive drugs, such as corticosteroids, other traditional immunosuppressive or cytotoxic agents and antifibrotics. Despite the high prevalence of fibrosis and its enormous impact on human health, there are currently no FDA-approved agents that can prevent, arrest, or reverse fibrosis.

Accordingly, what is needed in the art are new compounds and treatments for PFD generally and specifically PAH, for example.

### SUMMARY OF THE INVENTION

It has now been discovered that apyrase agents of the instant invention are useful for treating fibroproliferative diseases (“FPD”).

In one embodiment, the FPD that is treated according to the present invention is pulmonary hypertension (“PH”).

In one embodiment, the PH that is treated according to the present invention is pulmonary arterial hypertension (“PAH”).

In one embodiment, the PAH that is treated according to the present invention is secondary PAH, optionally associated with immunodeficiency or immunocompromise.

In one embodiment, the PAH that is treated according to the present invention is associated pulmonary and/or myocardial fibrosis.

In one embodiment, the FPD that is treated according to the present invention is associated right ventricular failure.

In one embodiment, the PFD that is treated according to the present invention is posthrombotic syndrome associated with venous thrombosis.

In one embodiment, the PFD that is treated according to the present invention is induced by tissue injury, chemical exposure, infection, inflammation, immune disregulation, or tissue rejection.

In one embodiment, methods and kits are provided for (i) assessing the efficacy of apyrase agent treatment of pulmonary hypertension; (ii) identifying subjects with pulmonary hypertension; and (3) assessing disease progression, by quantifying cytokines (set forth in Table 1-Table 3) in biological fluids.

### BRIEF DESCRIPTION OF THE DRAWINGS

FIG. 1 depicts the effect of EN Apyrase on pulmonary arterial pressure, systolic pressure, ventricular hypertrophy, and body weight.

FIG. 2 depicts the effect of EN Apyrase on pulmonary arterial patency

FIG. 3 depicts the attenuation of EN apyrase on formation of plexiform lesions in lung.

FIG. 4 depicts the effect of EN apyrase on collagen deposition in the lungs and right ventricle of the heart.

FIG. 5 depicts the restoration of CD39 and CD73 expression by EN apyrase in the lungs.

FIG. 6 depicts the effects of EN apyrase on fibrotic score after venous thrombosis in mice.

FIG. 7 depicts the effects of EN apyrase on vein wall thickening after venous thrombosis in mice.

### DETAILED DESCRIPTION OF THE INVENTION

As used here, the following definitions and abbreviations apply.

“Apyrase agent” (or “instant apyrase agent”) means an apyrase agent useful according to the present invention. “Apyrase agent” also includes homologs of known agents as discussed below.

“BALE” means bronchioalveolar lavage fluid.

“Examplary” (or “e.g.” or “by example”) means a non-limiting example.

“PAH” means pulmonary arterial hypertension.

“PFD” means fibroproliferative disease.

“PTS” means posthrombotic syndrome.

“Treat” means a therapeutic or prophylactic action that either prevents or reduces at least one clinically relevant index in an individual or in a population. “Treat”, in the context of treating bleeding, includes a reduction in likelihood of local hemorrhage at the site of injury to the vasculature as well as in adjacent and distal tissue.

### Apyrase Agents of the Present Invention

Apyrase agents of the present invention are useful for treating fibrosis (also referred to here as fibroproliferative disease) that is associated with physical, mechanical, pharmacological, infectious, or biological injury.

An apyrase agent can be any apyrase agent that has ATPase and ADPase activities. By ATPase activity, it is meant the activity that catalyzes the hydrolysis of phosphoanhydride bonds of adenosine triphosphate (ATP) to adenosine monophosphate (AMP) using two molecules of water (Reaction 1). By ADPase activity, it is meant the activity that catalyzes the hydrolysis of adenosine diphosphate (ADP) to AMP using one molecule of water (Reaction 2).

Reaction 1: ATP+2.H₂O→AMP+2 phosphate. Reaction 2: ADP+H₂O→AMP+phosphate

Certain apyrase agents can also hydrolyze other nucleoside triposphates such as GTP, CTP, UTP, and other nucleoside diphosphates such as GDP, CDP, and UDP with various substrate specificities or preferences. The hydrolysis reactions catalyzed by apyrase agents require either calcium or magnesium as co-factor.

Apyrase agents can belong to one of several well-known families of apyrases. For example, an apyrase agent can belong to the CD39 class. By way of example, an apyrase agent can be a CD39L class agent (e.g. L1-L10). The CD39 apyrase agent can be soluble, e.g. sol CD39 or sol CD39L3. Genera and species of useful agents are described by Chen et al. (U.S. Pat. No. 7,247,3000) and described by Jeong et al (U.S. Pat. No. 7,390,485). Additionally, apyrase agents include EN-apyrases taught in U.S. provisional patent application Ser. No. 61/294,695 filed on 13 Jan. entitled: “Therapeutic Apyrase Constructs, Agents, and Production Methods”, hereby incorporated by reference in its entirety. Apyrase agents include apyrase homologs.

An apyrase agent can be a soluble calcium-activated nucleotidase (SCAN gi 20270339; SCAN-1 gi: 22218108; EC 3.6.1.6) as described by Smith et al. (Arch. Biochem. Biophys., [2002], 406: 105-115). Such agents have sequence homology with the bed bug Cimex lectularius apyrase (gi: 4185746) (Valenzuela et al. J. Biol. Chem., [1998], 273:30583-305900).

An apyrase agent can be a 5′-nucleotidase (gi: 33520072; EC 3.1.3.5), for example, as found in humans. Such agents have sequence homology with the mosquito Aedes aegypti apyrase (gi: 1703351) (Champagne et al. Proc. Natl. Acad. Sci. USA, [1995], 92:694-698).

Useful apyrase agents can be an inositol polyphosphate 5′-phosphatase (gi: 346209; EC 3.1.3.56), for example, as occur in humans. Such agents have sequence homology with the Rhodnius prolixus apyrase (gi; 1546841) (Sarkis et al. Bicohem. J., [1986], 233:885-891).

An apyrase agent can be an agent modified to increase the ATPase or ADPase activities, thereby increasing the therapeutic activity according to the present invention. Examples of such agents (or “homologs”) are sol CD39L3 R67G, sol CD39L3 R67G T69R (SEQ ID No:3), and sol CD39L3 T69R, as taught in Chen et al. (U.S. Pat. No. 7,247,300) and in Jeong et al (U.S. Pat. No. 7,390,485).

“Homologs”, in reference to apyrase agents, are agents that have (1) structural similarity to a class of apyrase (e.g. as discussed above, CD39; SCAN, SCAN-1,5′-nucleotidase, inositol polyphosphate 5′-phosphatase); (2) ADPase and ATPase activity; and (3) one or more substitutions (i.e. differences) from wild type apyrase. By “structurally similar” it is meant about or more than about any of 80% or 90% or 95% homology. Moreover, conservative substitutions, as they are now commonly known in the art, are expressly contemplated.

### Use of Apyrase Agents to Treat Fibrosis and Fibroproliferative Disease

According to the present invention, apyrase agents are useful for treating fibrosis (or fibroproliferative disease).

Fibrosis that can be usefully treated by an apyrase agent includes chronic fibrosis. Chronic fibrosis can be any chronic fibrosis such as fibrosis of any major organs such as lung, liver, kidney, heart, digestive system, and skin.

Fibrosis that can be usefully treated by an apyrase agent includes acute fibrosis. Acute fibrosis can be any acute fibrosis (e.g. with a sudden and severe onset and of short duration) that occurs, e.g., as a common response to various forms of trauma including injuries, ischemic illness (e.g., cardiac scarring following heart attack), environmental pollutants, alcohol and other types of toxins, acute respiratory distress syndrome, radiation and chemotherapy treatments. Any tissue damaged by trauma can become fibrotic (e.g. especially if the damage is repeated) and usefully treated with the instant apyrase agents.

Apyrase agents of the instant invention are useful for treating “interstitial fibrosis”; i.e. fibrosis relating to or situated in the small, narrow spaces between tissues or parts of an organ. By way of example, apyrase agent can be used to treat interstitial pulmonary fibrosis (also known as interstitial lung disease and pulmonary fibrosis), i.e. fibrosis (or scarring) of the interstitium (the tissue between the air sacs of the lungs). Apyrase agent can be used to treat renal interstitial fibrosis (also known as kidney fibrosis), characterized by the destruction of renal tubules and interstitial capillaries and/or by the accumulation of extracellular matrix proteins.

Apyrase agents of the instant invention are useful for treating “vascular remodeling”, a type of fibrosis that refers to the active process of structural and cellular changes in the vasculature. Typically, this type of fibrosis is further characterized by an increased number of cells which express alpha-smooth muscle actin, possibly resulting from accumulation of alpha-smooth muscle positive cells from the proliferative expansion of resident vascular smooth muscle cells (SMC), recruitment of circulating progenitor cells to sites of vascular injury, or transition of endothelial cells towards a mesenchymal phenotype (EnMT). Examples of fibrotic vascular diseases that can be treated by apyrase agents according to the instant invention include, e.g., cardiac disease, cerebral disease, and peripheral vascular disease, and with organ failure in a variety of chronic diseases affecting the pulmonary system.

Exemplary fibroproliferative diseases that can usefully be treated by the instant apyrase agents include scleroderma (including morphea, generalized morphea, or linear scleroderma), kidney fibrosis (including glomerular sclerosis, renal tubulointerstitial fibrosis, progressive renal disease or diabetic nephropathy), cardiac fibrosis (e.g., myocardial fibrosis), pulmonary fibrosis (e.g., glomerulosclerosis pulmonary fibrosis, idiopathic pulmonary fibrosis, silicosis, asbestosis, interstitial lung disease, interstitial fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fascilitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, glioblastoma in Li-Fraumeni syndrome, sporadic glioblastoma, myleoid leukemia, acute myelogenous leukemia, myelodysplastic syndrome, myeloproliferative syndrome, gynecological cancer, Kaposi's sarcoma, Hansen's disease, collagenous colitis, acute fibrosis, systemic sclerosis, and fibrosis arising from tissue or organ transplant or graft rejection.

Although many different types of tissues and organs can develop fibroproliferative disease, they all are caused by a common mechanism, i.e. the accumulation of fibrotic material at various tissues and organs. The formation or development of excess fibrous connective tissue in an organ or tissue can be a reparative or reactive process, as opposed to a formation of fibrous tissue as a normal constituent of an organ or tissue. Fibrotic conditions can include excessive amounts of extracellular matrix accumulation within a tissue, forming tissue which causes dysfunction and, potentially, organ failure. Fibroproliferative diseases treated according to the present invention typically have increased myofibroblast activation.

Posthrombotic Syndrome Associated with Venous Thrombosis

In one embodiment, the FPD that is usefully treated by apyrase agents according to the present invention is posthrombotic syndrome (PTS) associated with venous thrombosis. PTS is defined clinically by the presence of leg swelling, venous claudication, pain, and ulceration. PTS is characterized by blood reflux caused by compromised vein valves, outflow obstruction, and tissue hypoxia that is secondary to vein wall thickening and fibrosis. PTS is characterized by a fibrotic vein injury following deep vein thrombosis. Fibrosis, a consequence of chronic inflammation, is a result of fibroblast or smooth muscle cell activation and collagen deposition in the thrombosed area (Wojicik B M et al. Interleulin-6: a potential target for post-thrombotic syndrome. Ann Vasc Surg. 25: 229-239, 2011).

The vein wall response is initiated early following thrombus formation and persists even in the presence of total resolution. Through insight of the inventor, apyrase agent treatment of PTS is markedly superior to current anticoagulant therapy because, in addition to preventing clotting without bleeding risks associated with other anticoagulents, it prevents the intraluminal vein wall scarring, fibrosis, and stiffness, thereby reducing or eliminating post-thrombotic venous obstruction.

### Use of Apyrase Agents to Treat PH Diseases and Associated Heart Failure

In one embodiment, the FPD that is usefully treated by apyrase agents according to the present invention is pulmonary hypertension (PH), especially forms of where hypertension is due, in part, to microthrombus or fibrosis.

In one embodiment, the PH usefully treated here is PH of the World Health Organization (WHO) group 1. This is a group of diseases also known as pulmonary arterial hypertension (PAH) and characterized by elevated pulmonary arterial pressure and elevated blood flow resistance due to a precapillary pulmonary microangiopathy.

Apyrase agents, according to the present invention, are useful for treating primary and secondary PAH. Examples of such secondary PAH are forms that result from pulmonary or cardiac disorders, wherein the pathogenesis may involve mechanisms of hypoxic vasoconstriction, decreased area of the pulmonary vascular bed and volume/pressure overload.

Among the forms of secondary PAH that can usefully be treated by apyrase agent according to the present invention include Idiopathic, heritable, connective tissue disease, human immunodeficiency virus infection, portopulmonary hypertension, congenital heart disease, drug/toxin induced, chronic hemolytic anemia, schistosomiasis, persistent PH of the newborn, pulmonary veno-occlusive disease and pulmonary capillary hemangiomatosis.

Among the forms PAH that can usefully be treated by apyrase agent according to the present invention include forms where immunodeficiency serves a role in pathophysiology and those forms where it does not.

Without being bound by theory, the applicants believe that apyrase is effective in PAH at least because it mitigates the pathophysiologic role of ATP in two different processes. In such patients, there is an underlying systemic inflammation (mediated in part by extracellular ATP and ADP) that can arise through pulmonary arterial endothelial cell (PAEC) apoptosis or immune disregulation, leading to occlusion of the pulmonary artery lumen and restriction of blood flow. Extracellular ATP and ADP mediated inflammation in PAH can also promote pulmonary arterial smooth muscle cell (PASMC) proliferation and hypertrophy, leading to thickening of the pulmonary artery wall and restriction of blood flow. Thus, apyrase agent's action to reduce extracellular ATP and ADP can block PAEC apoptosis and micothrombosis while simultaneously blocking PASMC proliferation and fibrosis—two pathological processes highly implicated in PAH pathogenesis.

### Therapeutic Compositions and Administration of Apyrase Agents

The present invention provides compositions comprising a biologically effective amount of apyrase agent or biologically active derivative in a pharmaceutically acceptable dosage. Therapeutic composition of apyrase agents or biologically active derivatives (i.e. homologs) may be administered clinically to a patient before symptoms, during symptoms, or after symptoms.

Administration of apyrase agents to achieve therapeutic effect may be given by oral, transdermal, transmucosal, inhalation, or parenteral administration. Parenteral administration can be, e.g., by intraveneous injection such as bolus injection, continuous infusion, sustained release, or other pharmaceutically acceptable techniques. Certain clinical situations may require administration of apyrase agents as a single effective dose, or may be administered as multiple doses.

Apyrase agents can be administered by injecting a bolus into the subcutis (i.e., a subcutaneous injection).

It should also be recognized by the skilled artisan that alternative formulations of apyrase agents can allow for alternate routes of administrations (e.g. oral [e.g. enteral, sublabial, or respiratory], ophthalmic, otologic, nasal, rectal, or dermal).

Optionally apyrase agents are administered to patients in a pharmaceutically acceptable form containing physiologically acceptable carriers, excipients or diluents. Such diluents and excipients may be comprised of neutral buffered saline solution, antioxidants (for example ascorbic acid), low molecular weight polypeptides (for example polypeptides 5≦10 amino acids) amino acids, carbohydrates (for example, glucose, dextrose, sucrose, or dextrans), chelating agents such as EDTA, stabilizers (such as glutathione). Additionally, cosubstrates for the apyrase agents, for example, calcium (Ca²⁺) may be administered at time of dosage for maximal activity of the enzyme. Such carriers and diluents are nontoxic to the patient at recommended dosages and concentrations.

It is also envisioned in the present invention that apyrase agents may be administered with other agents that unexpectedly enhance the benefit of apyrase agent alone. For example, it is envisioned that administration of other antiplatelets or anticoagulants, such as aspirin, heparin or bivalirudin with apyrase agents or biologically active derivative may have additional benefits such as improve reperfusion, extend therapeutic time window, prevent reocclusion, and prevent vascular thrombosis. It is also envisioned that administration of apyrase agents may improve efficacy and lower the effective dosage of thrombolytics (tissue plasminogen activator (tPA), vampire bat plasminogen activator, urokinase, streptokinase, staphylokinase, and ancrod) or immunosuppressants. It is still further envisioned in the present invention that operable fusion polypeptides between, for example, an ADP enhanced apyrase agent and thrombolytic (for example, tPA) may provide an ideal therapeutic solution for acute myocardial infarction (AMI), percutaneous coronary intervention (PCI) and acute ischemic stroke (AIS).

Dosage requirements of apyrase agents may vary significantly depending on age, race, weight, height, gender, duration of treatment, methods of administration, biological activity of apyrase agents, and severity of condition or other clinical variables. Effective dosages may be determined by a skilled physician or other skilled medical personnel.

### Treatment Regimen.

Through insight of the inventor, it has been discovered that optimally effective treatment regimens of pulmonary hypertension and fibrotic diseases typically requires multiple treatments over the course of several weeks (e.g. 2 to 4) or several months (e.g. 2 to 6 or more).

Administration of apyrase agent is generally at a dose of 0.1-10 mg/kg, more typically at a dose of 0.2-5 mg/kg. The frequency of dosing is typically weekly to monthly.

Apyrase agent, according to the present invention, is administered intravenously in a solution at a concentration of 5 to 20,000 mg/liter.

Administration of apyrase for pulmonary hypertension and fibrotic diseases typically is performed as an adjunct to therapy consisting of one or more of vasodilators, e.g. a β-blocker, a prostacyclin analog, an endothelin receptor antagonist, a phosphodiesterase-5 inhibitor, or a guanylyl cyclase stimulator.

### Diagnosis of Pulmonary Hypertension

In one embodiment, a method is provided for assessing the efficacy of apyrase agent treatment of pulmonary hypertension, for identification of subjects with pulmonary hypertension, or for assessing disease progression. According to the instant invention, one or more biological samples are obtained from the patient from, e.g., plasma, bronchioalveolar lavage fluid (BALF), or pulmonary and RV tissue lysates. One or more cytokines from Table 1 are quantified in BALF and compared to normal subjects, other subjects with pulmonary hypertension, or to serial samples obtained from the same (affected) subject, As taught herein, reduction of one or more of these cytokines from Table 1, when compared to normal subjects, is evidence of pulmonary hypertension. An increase in one or more cytokines from Table 1 is evidence of therapeutic efficacy of an apyrase agent or other therapeutic agent. A further decrease in concentrations of such cytokines are evidence of disease progression.

TABLE 1
Cytokines

GM-CSF
IL-1B
IL-2
IL-10
IL-12p70
IL-18
FRACTALKINE
VEGF
TNFalpha

TABLE 2
Cytokines

IFN gamma
RANTES

TABLE 3
Cytokines

G-CSF
EOTAXIN
GM-CSF
IL-Ia
IL-4
IL-1B
IL-2
IL-6
IL-10
IL-12p70
IL-5
IL-17A
MCP-1
VEGF
MIP-2
RANTES

One or more cytokines from Table 2 are quantified in BALF and compared to normal subjects, other subjects with pulmonary hypertension, or to serial samples obtained from the same (affected) subject, As taught herein, an increase of one or more of these cytokines from Table 2, when compared to normal subjects, is evidence of pulmonary hypertension. A decrease in one or more cytokines from Table 2 is evidence of therapeutic efficacy of an apyrase agent or other therapeutic agent. A further increase in concentrations of such cytokines are evidence of disease progression.

One or more cytokines from Table 3 are quantified in plasma (or serum) and compared to normal subjects, other subjects with pulmonary hypertension, or to serial samples obtained from the same (affected) subject, As taught herein, an increase of one or more of these cytokines from Table 3, when compared to normal subjects, is evidence of pulmonary hypertension. A decrease in one or more cytokines from Table 3 is evidence of therapeutic efficacy of an apyrase agent or other therapeutic agent. A further increase in concentrations of such cytokines are evidence of disease progression.

### EXAMPLES

The following examples are intended to illustrate but not to limit the invention. Moreover, scientific discussions below of underlying mechanisms gleaned from the data are also not meant as limitations of the inventions described here.

### Example 1

### Apyrase Agent Preparation

Apyrase agent used in these examples is a soluble apyrase agent made from a construct coding for CD39L3 (e.g. SEQ ID No: 1), absent about 43 amino acids sequence from the N-terminus and absent about 44 amino acid sequence from the C-terminus corresponding to the membrane spanning domains as described by Jeong et al (U.S. Pat. No. 7,390,485). The apyrase further contains a substitution of an arginine for a glycine at residue 67 and substitution of a threonine for an arginine at residue 69 (where the residue number refers to the CD39L3 (SEQ ID No: 1).

Useful constructs can be derived, in part, from SEQ ID No: 2 which codes for CD39L3.

The apyrase construct further contains a sequence encoding bovine α-lactalbumin signal peptide sequence.

The apyrase construct was transformed into a Chinese Hamster Ovary (CHO) cell lines by retrovector.

Conditioned medium was harvested from the transformed CHO cells and apyrase agent was purified by two-ion exchange chromatography steps (ANX and SP). Analysis of the N terminus of apyrase agent by this method revealed the following amino acids: Glu-Val-Leu-Pro-Pro-Gly-Leu-Lys-Tyr-Gly-Ile.

Additional details on methods of production of the apyrase agent used in these examples are found in U.S. application Ser. No. 13/522,311. Accordingly, adopted herein is the term the “EN-apyrase” as claimed and taught therein and as produced and used below.

### Example 2

### Apyrase Agent for Treating PAH and Associated Fibrosis

This study used a clinically relevant model of PAH in rats to determine whether EN apyrase treatment reverses the PAH disease. The combination treatment with hypoxia and SU5416 causes the initial apoptosis of lung endothelial cells, followed by the selection of phenotypically altered apoptosis-resistant endothelial cells. The oxidative stress and prominent inflammatory state lead to severe, angio-obliterative pulmonary hypertension and associated right ventricular failure. Occlusive neointimal and plexiform lesions are formed in small peripheral pulmonary arteries, resembling the pathology of human PAH patients.

Method. Six-week-old male Sprague-Dawley rats, were divided into 7 groups: Normoxia (Group #1, n=3); Hypoxia (Hx, n=4) (Group #2 and 3, simulated altitude of 18,000 ft for 3 weeks and returned to normoxia for 2 or 3 weeks); Hypoxia plus SU5416 (HX+SU) (single subcutaneous injection of 20 mg/kg at day 1 and exposed to hypoxia for 3 weeks and returned to normoxia for 2 or 3 weeks (Group #4 and 5) (HX+SU, n=6); Group #6 HX+SU rats treated with EN apyrase (1 mg/kg, 3× per week for 3 weeks starting on week 3, n=6); Group #7 HX+SU rats treated with EN apyrase (1 mg/kg, 3× per week for 4 weeks starting on week 3, n=6). The right lungs were snap-frozen for protein and RNA isolation, and the left lung was inflated with 0.5% low-melting agarose and formalin fixed for immunohistochemical evaluation. Pulmonary arterial pressures and cardiac output were measured by inserting a pulmonary arterial catheter. Hematocrit and right ventricular hypertrophy (the ratio of right ventricle mass over left ventricle plus septum mass: RV/(LV+S) were determined. Expression levels of CD39 and CD73 were determined by immunohistochemistry and Western blot analysis.

Apyrase Agent Reversed Both Pulmonary and RV Functions.

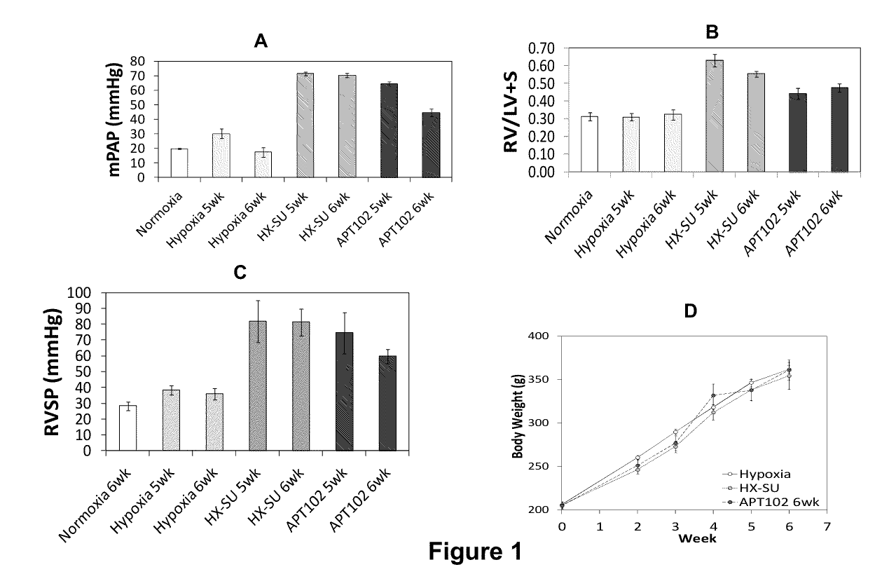

SD rats treated with hypoxia for 3 weeks had increased mean pulmonary arterial pressures (PAP) which remained higher than the baseline upon re-exposure to normoxia for 2 weeks (week 5, FIG. 1A). The mean PAP fully returned to normal on week 6. In contrast, rats of HX+SU developed irreversible PAH as reported previously (Taraseviciene-Stewart L et al. Inhibition of the VEGF receptor 2 combined with chronic hypoxia causes cell death-dependent pulmonary endothelial cell proliferation and severe pulmonary hypertension. FASEB J. 15: 427-438, 2001.

The mean PAP was 71.47±1.16 mmHg on week 5 and remained high on week 6 (70.4±1.64 mmHg). EN apyrase treatment modestly reduced the mean PAP (p=0.07) on week 5 and dramatically reversed to 44.6±2.78 mmHg on week 6 which is significantly improved compared to both HX+SU and EN apyrase 5 week groups. Hypoxia alone did not cause right ventricular hypertrophy (FIG. 1B). In contrast, pronounced right ventricular hypertrophy was induced with hypoxia plus SU. The ratio of RV/(LV+S) was 0.63±0.04 versus 0.31±0.02 in the hypoxia group. The ratio on week 6 was not statistically different from that on week 5. EN apyrase treatment significantly attenuated the ratio to 0.44±0.03 and 0.47±0.02 on weeks 5 and 6 respectively. The RV systolic pressure (RVSP) was significantly elevated in the HX+SU−treated rats. EN apyrase reduced RVSP by 26% (FIG. 1C, p=0.08) and RV diastolic pressure by 33% (data not shown) on week 6.

Apyrase Agent Reversed/Attenuated Pathological Vascular Remodeling and Fibrosis.

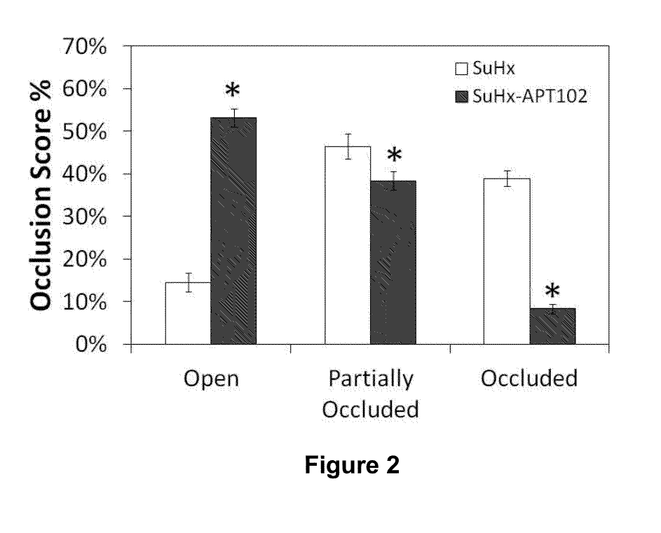

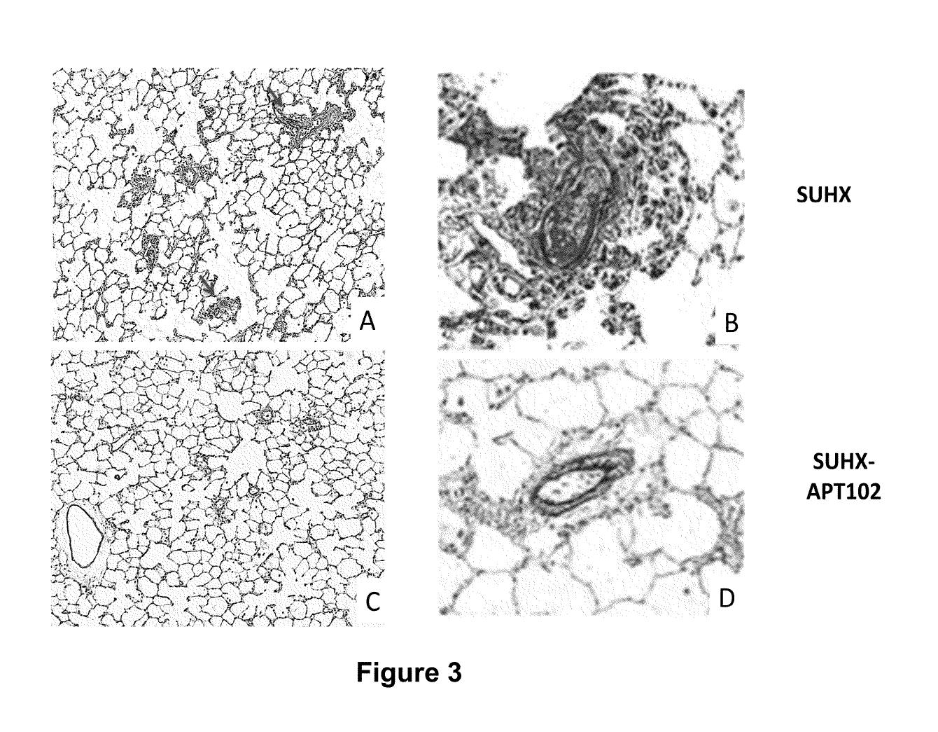

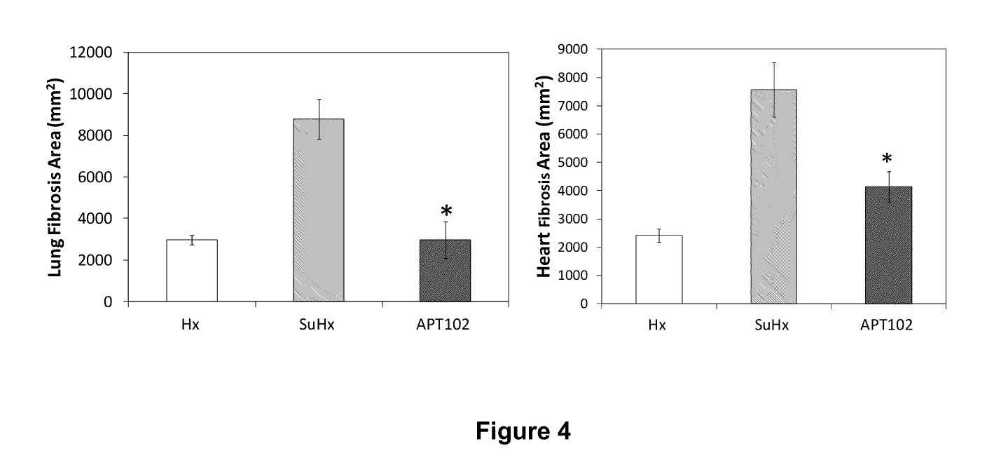

Treatment with EN apyrase substantially improved the pulmonary artery patency with 53%, 39% and 8% arteries that are open, partially occluded and occluded, respectively, vs 14%, 47% and 39% for the HX+SU group (FIG. 2). Lungs of HX+SU groups showed typical plexiform lesions characterized by almost complete luminal occlusion of medium-sized and precapillary intra-alveolar arteries (FIGS. 3A and B). In contrast, EN apyrase-treated rat lung did not show any plexiform lesions (FIGS. 3C and D). Compared to the hypoxia group, pronounced fibrosis was obvious in both lung and RV of the HX+SU group (FIG. 4). Both mucopolysaccharide (data not shown) and collagen deposition, were effectively attenuated with EN apyrase treatment (FIG. 4).

Apyrase Agent Restored CD39 and CD73 Expression.

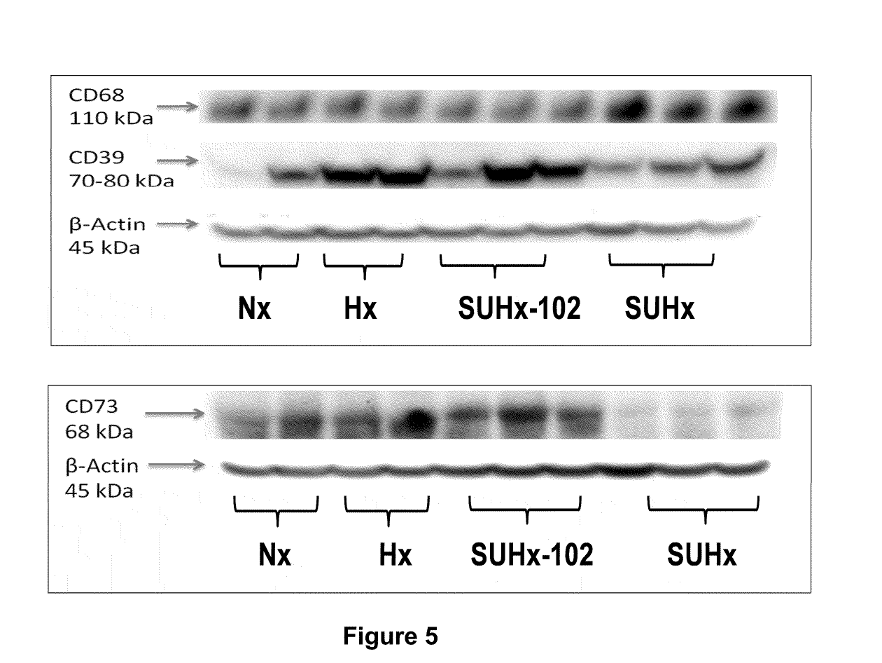

Western blot analysis shows that CD39 expression was enhanced in the hypoxic lung extracts but reduced in the HX+SU group (FIG. 5A), which is likely due to oxidative stress and inflammation. Treatment with EN apyrase restored the expression level which may be due to attenuation of inflammatory state. The expression of CD73 was significantly downregulated in the HX+SU group when compared to Hx alone or to control lung extracts (FIG. 5B). Treatment with EN apyrase restored CD73 levels. On the other hand, HX+SU increased macrophage CD68, a marker of inflammation (FIG. 5A). EN apyrase restored the level of CD68 to normal. Immunohistochemical staining also showed that HX+SU decreased CD39 levels on tissue-infiltrating macrophages (Data not shown) while EN apyrase restored the expression of CD39 that is important for macrophage differentiation into regulatory anti-inflammatory M2 phenotype.

EN apyrase was safe and did not cause body weight loss (FIG. 1D) or mortality. There were no significant changes in heart rate or blood pressure.

To determine the clinical relevance of the suppressed CD39/CD73 pathway in the HX+SU model, we examined the lungs of 2 healthy and 4 PAH human patients. CD39 was expressed in the healthy lung tissues. However, CD39 expression was severely suppressed around occluded vascular lumens of plexiform lesions in all four PAH lungs.

### Conclusion

EN apyrase is safe and effective in reversing pulmonary and RV remodeling and function. The restoration of the endogenous CD39/CD73 pathway with EN apyrase is expected to generate profound and lasting outcomes to maintain homeostasis. Hence, EN apyrase-based therapy is safe and effective for PAH treatment.

### Example 3

### Apyrase Agent for Treating Secondary Pulmonary Hypertension Associated with Immunocompromise

The purpose of this study is to demonstrate that apyrase agent can reverse pulmonary and right ventricular remodeling and function and improve survival in secondary pulmonary hypertension associated with immunocompromise in a 12 weeks study using the SU5416/athymic rat model. The key milestone is to reverse/attenuate mean PAP and RVEF by 50% at 12 weeks post EN apyrase treatment, compared to the vehicle (p<0.05).

Experimental Design:

80 rats (n≧12/group for groups 1-4) are randomized to 4 treatment groups (n=20/group with anticipated mortality of 40% for the control and treatment groups). Six non-treated and age-matched rats (Group 5) will serve as healthy rat controls. Group 1: Vehicle (i.v.); Group 2: EN apyrase at 1 mg/kg, 3 times a week for 4 weeks; Group 3: EN apyrase at 0.3 mg/kg, 3 times a week for 4 weeks; Group 4: EN apyrase at 3 mg/kg, 3 times a week for 4 weeks. Group 5: Healthy rat controls. Treatment is initiated at 21 days after SU5416 injections. The primary efficacy endpoint is RVEF (19 weeks); the primary safety endpoint is mortality (weekly). The secondary endpoints include: exercise endurance (19 weeks), body weight (weekly), mPAP (baseline, 3, 7, 19 weeks), cardiac output (baseline, 3, 7, 19 weeks); RVSP (baseline, 3, 7, 19 weeks); RV dilatation (baseline, 3, 7, 19 weeks); RV hypertrophy (19 weeks), RV fibrosis (19 weeks), RV capillary volume/density (19 weeks), collagen deposition (19 weeks), CD39 and CD73 expression (19 weeks), and LTB4 (19 weeks).

Results.

Apyrase agent treatment in this study restores CD39 expression and facilitates the phenotypic change of macrophage from pro- to anti-inflammatory. VSMC proliferation and inflammation is profoundly attenuated. Moreover, apyrase agent significantly reverses/attenuates adverse vascular remodeling and improves both cardiac and pulmonary function and survival.

### Example 4

### Apyrase Agent for Treating PAH in Humans

Subjects are screened for PAH with a Swan-Ganz catheter through the right side of the heart. Physical exams are conducted to assess PAH signs and symptoms such as a widely split S2 or second heart sound, a loud P2 or pulmonic valve closure sound, sternal heave, pulmonary regurgitation, peripheral edema (swelling of the ankles and feet), ascites (abdominal swelling due to the accumulation of fluid), hepatojugular reflux, and clubbing. Blood analysis is conducted to assess whether there is an elevation in IL-18 levels.

Subject with pulmonary artery pressure exceeding 25 mm Hg (3300 Pa) at rest or exceeding 30 mm Hg (4000 Pa) after exercise and with one or more of the aforementioned signs and symptoms of PAH are included in the study.

Included subjects receive apyrase therapy administer by I.V. infusion of 0.001 to 1 liter of normal saline containing 5, 50, 100, or 200 mg EN apyrase per liter such that subjects receive 0.1, 0.4, 0.8, or 1.6 mg/kg body weight. Apyrase therapy is repeated every two weeks. After 10 weeks, pulmonary artery pressure returns into or towards the normal range and there is a substantial reversal in IL-18 in BALF. Pulmonary regurgitation, peripheral edema, ascites, and hepatojugular reflux is absent or markedly reduced. At this time (10 weeks), apyrase therapy is continued every two months and bi-annual exams are conducted. If signs and symptoms of PAH return, the frequency of apyrase administration is increased.

### Example 5

### Apyrase Agent Co-Administrations for Treating Pulmonary Hypertension

In this study, apyrase agent efficacy in the PAH rat is compared to that of bostentan alone, carvelidol alone, apyrase agent—boststenan co-therapy, and to apyrase agent—carveldio co-therapy.

The key milestone is to reduce mPAP or improve RVEF by 30% at 12 weeks post therapy, compared to bosentan or carvedilol (p<0.05).

Experimental Design:

120 rats (n≧12/group) are randomized to 6 treatment groups (n=20/group for group 1-6 with anticipated mortality of 30-40% for the control and treatment groups). Six non-treated and age-matched rats will serve as healthy rat controls with or without exposure to hypoxia (Group 7 and 8): Group 1: Vehicle (i.v.); Group 2: EN apyrase at dose to be determined in Aim 1, 3 times a week for 4 weeks; Group 3: Bosentan at 250 mg/kg mixed with rodent dough food for weeks; Group 4: Carvelidol at 15 mg/kg, once daily per oral gavage for 16 weeks; Group 5: EN apyrase for 4 weeks plus Bosentan for 16 weeks; Group 6: EN apyrase for 4 weeks plus Carvelidol for 16 weeks; Group 7: non-treated and age-matched rats to be kept at normoxia; Group 8: age-matched rats to be exposed to hypoxia for weeks.

Treatment is initiated at 21 days post HX+SU exposure. The primary efficacy endpoint is RVEF (19 weeks); the primary safety endpoint is mortality (weekly). The secondary endpoints include: exercise endurance (19 weeks), body weight (weekly), mPAP (baseline, 3, 7 19 weeks), cardiac output (baseline, 3, 7, 19 weeks); RVSP (baseline, 3, 7, 19 weeks); RV dilatation (baseline, 3, 7, 19 weeks); RV hypertrophy (19 weeks), RV fibrosis (19 weeks), RV capillary volume/density (19 weeks), collagen deposition (19 weeks), and CD39 and CD73 expression (19 weeks).

Results

The EN apyrase treatment is safer and more effective than bosentan or carvelidol in reversing/attenuating adverse vascular remodeling and improving both cardiac and pulmonary function and survival. Apyrase—bostentan cotherapy and Apyrase agent—carvelidol co-therapy is more effective than monotherapy.

### Example 6

### Effects of Apyrase Agent on Cytokine Homeostasis

Cytokines were examined in plasma and bronchioalveolar lavage fluid (BALF) in the rat PAH study of Example 2.

Cytokines in Plasma.

Treatment with hypoxia or HX+SU exposure induced an overwhelming “cytokine storm” in plasma with increased concentrations of 2-30 fold for most of the cytokines (Table 4). Notable exceptions were IL18, IP-10, and Fractalkine, which did not change significantly. HX+SU group had more pronounced increases in IL1β, MCP-1, and Rantes, when compared to the HX group.

Treatment with EN apyrase partially or completely reversed the cytokine productions to the basal normoxic level in at least G-CSF, EOTAXIN, GM-CSF, IL-Ia, IL-4, IL-1B, IL-2, IL-6, IL-10, IL-12p70, IL-5, IL-17A, MCP-1, VEGF, MIP-2, and RANTES.

These results demonstrate that these cytokines (i.e. the cytokines of Table 3) are useful as indicators of PAH (when elevated), indicators of the usefulness of treatment with apyrase agent (when elevated), and indicators of the effectiveness of treatment with apyrase agent (when decreased with treatment).

Cytokines in BALF.

In contrast to the cytokine response in plasma, treatment of the rats with hypoxia or HX+SU exposure resulted in a substantial decrease in GM-CSF, IL-1B, IL-2, IL-10, IL-12p70, IL-18, FRACTALKINE, VEGF, and TNFalpha Table 5).

When En apyrase was administered to the PAH rat (HX+SU), there was a substantial reversal (decrease) in the levels of these same cytokines.

These results demonstrate that these cytokines (i.e. the cytokines of Table 1) are useful as indicators of PAH (when decreased in BALF), indicators of the usefulness of treatment with apyrase agent (when decreased in BALF), and indicators of the effectiveness of treatment with apyrase agent (when increased in BALE upon treatment with apyrase agent) Table 5.

Treatment of the rats with hypoxia or HX+SU exposure resulted in a substantial increase in IFNγ and RANTES. When En apyrase was administered to the PAH rat (HX+SU), there was a substantial decrease (normalization) of the levels of these same cytokines in BALF Table 5.

These results demonstrate that these IFN y and RANTES are useful as indicators of PAH (when increased in BALF), indicators of the usefulness of treatment with apyrase agent (when increased in BALF), and indicators of the effectiveness of treatment with apyrase agent (when decreased in BALF upon treatment with apyrase agent).

### Example 7

### Apyrase Agent for Treating Fibrosis and Posthrombotic Syndrome Associated with Venous Thrombosis

Electrical injury model of venous thrombosis was used for chronic fibrotic study (Diaz J A et al. Thrombogenesis with continuous blood flow in the inferior vena cava: a novel mouse model. Thromb Haemost. 104: 366-375, 2010; Diaz J A et al. The electrolytic inferior vena cava modle (EIM) to study thrombogenesis and thrombus resolution with continuous blood flow in the mouse. Thromb Haemost. 109: 1158-1169, 2013).

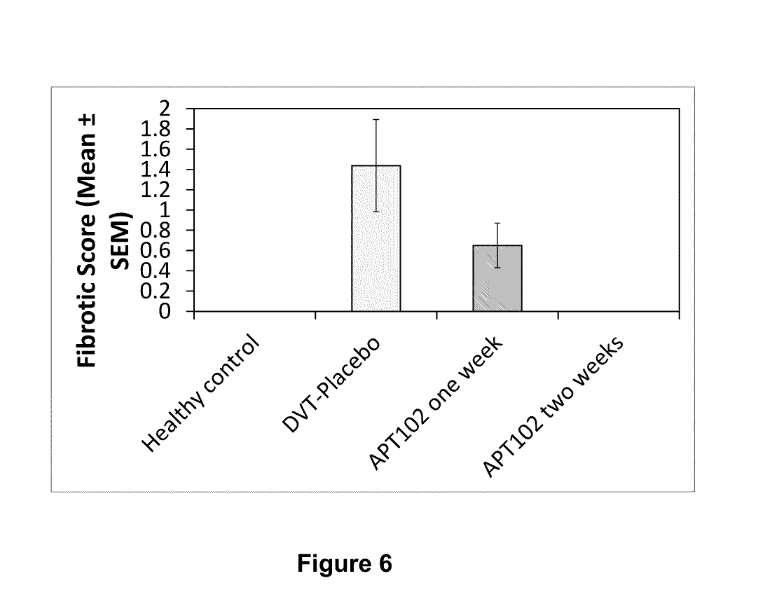

Mice were randomized and blinded into the four groups (n=5/group). Healthy control; placebo: Saline i.p. daily for 14 days; EN apyrase: 1.0 mg/kg i.p. bolus on day 1, 3, and 5 days (one week); EN apyrase: 1.0 mg/kg i.p. every 2 days for 14 days (two weeks). Collagen deposition was stained with Masson's trichrome 14 days post DVT induction. The model consistently induced IVC fibrosis in all mice with the mean increased collagen deposition by approximately 6-fold compared to the healthy mice (FIG. 6).

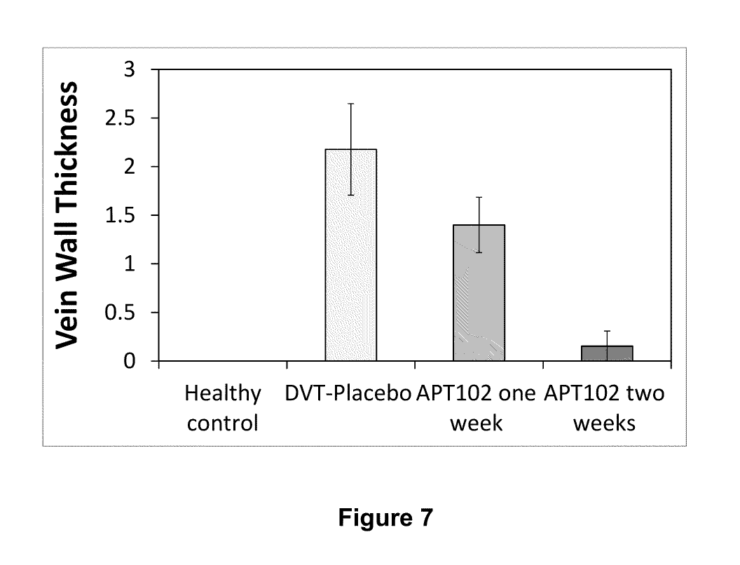

Treatment with EN apyrase for one or two weeks partially or almost completely attenuated fibrosis and vein wall thickening (FIG. 7). There was one death in the placebo group, while no death, increased bleeding or gross side effects were observed in the EN apyrase groups. These data suggest that EN apyrase is safe and may be effective for treatment of posthrombotic syndrome.

TABLE 4
Plasma cytokine analysis (6 wk, pg/ml))

| | Mean ± SEM | | | |
|---|---|---|---|---|
| | | | | Hx + SU EN + |
| | Nx | Hx | Hx + SU | apyrase |
| G-CSF | <1.28 | 5.39 ± 4.11 | 4.09 ± 1.60 | <1.28 |
| EOTAXIN | <3.70 | 10.44 ± 4.27 | 11.36 ± 1.16 | 7.16 ± 1.00 |
| GM-CSF | <3.47 | 9.06 ± 5.59 | 5.02 ± 1.55 | <3.47 |
| IL-Ia | <4.01 | 12.81 ± 8.80 | 11.74 ± 5.11 | <4.01 |
| LEPTIN | 1,662.50 ± 17.50 | 4,355 ± 2,258 | 2,546.50 ± 376.04 | 1,758 ± 282 |
| MIP-1a | 4.41 ± 1.12 | 10.85 ± 2.83 | 12.34 ± 3.74 | 7.00 ± 5.37 |
| IL-4 | <2.00 | 58.98 ± 23.69 | 41.75 ± 12.32 | 20.20 ± 4.22 |
| IL-1B | <0.73 | 5.45 ± 2.27 | 22.96 ± 14.17 | 5.07 ± 4.34 |
| IL-2 | 7.95 ± 2.56 | 26.86 ± 20.36 | 23.32 ± 6.85 | 7.62 ± 2.63 |
| IL-6 | <21.66 | 376.76 ± 250.97 | 238.75 ± 56.95 | <21.66 |
| EGF | 1.84 ± 0.41 | 2.77 ± 0.56 | 2.50 ± 0.42 | 0.74 ± 0.38 |
| IL-13 | <0.91 | 33.81 ± 20.12 | 33.47 ± 8.74 | 15.61 ± 3.67 |
| IL-10 | <2.25 | 15.50 ± 2.89 | 29.42 ± 7.16 | 5.44 ± 2.83 |
| IL-12p70 | 12.42 ± 2.69 | 42.60 ± 20.98 | 30.24 ± 6.55 | 15.68 ± 3.14 |
| IFNg | <4.52 | 31.30 ± 26.78 | 35.09 ± 12.96 | 14.71 ± 10.19 |
| IL-5 | 12.01 ± 1.83 | 94.02 ± 38.76 | 84.16 ± 16.76 | 49.45 ± 8.58 |
| IL-17A | <1.86 | 15.52 ± 10.25 | 10.59 ± 3.54 | 3.71 ± 1.10 |
| IL-18 | 144.26 ± 40.44 | 120.06 ± 16.97 | 151.81 ± 46.46 | 57.71 ± 34.30 |
| MCP-1 | 490.31 ± 59.03 | 684.74 ± 227.22 | 1,030.01 ± 109.34 | 618.50 ± 189.87 |
| IP-10 | 181.83 ± 40.58 | 174.99 ± 21.67 | 199.70 ± 36.73 | 132.21 ± 24.85 |
| GRO/KC | <9.84 | 27.57 ± 17.73 | 32.90 ± 14.40 | 41.80 ± 31.96 |
| VEGF | 50.18 ± 24.19 | 67.55 ± 2.86 | 87.97 ± 16.67 | 39.07 ± 11.27 |
| Fractalkine | 38.58 ± 1.50 | 33.25 ± 11.37 | 23.80 ± 2.19 | 17.48 ± 4.30 |
| LIX | 372.67 ± 129.43 | 1,467.23 ± 678.77 | 1,913.51 ± 769.71 | 1,181.48 ± 966.42 |
| MIP-2 | <16.36 | 28.51 ± 12.15 | 27.91 ± 4.52 | 17.34 ± 0.98 |
| TNFa | <1.45 | 1.86 ± 0.41 | 1.49 ± 0.04 | <1.45 |
| RANTES | 39.46 ± 24.48 | 273.42 ± 78.95 | 391.92 ± 85.88 | 206.91 ± 121.38 |

TABLE 5
Cytokines in BALF (6 wk, μg/ul)

| Cytokine | | Hx + SU | Hx + SU + |
|---|---|---|---|
| (ug/uL) | Hx | (PAH) | EN apyrase |
| G-CSF | 0.10 ± 0.03 | 0.11 ± 0.05 | 0.08 ± 0.02 |
| EOTAXIN | 0.33 ± 0.08 | 0.28 ± 0.11 | 0.28 ± 0.08 |
| GM-CSF | 0.51 ± 0.13 | 0.14 ± 0.01 | 0.50 ± 0.14 |
| IL-1A | 0.79 ± 0.14 | 0.73 ± 0.33 | 0.51 ± 0.11 |
| LEPTIN | 4.50 ± 1.18 | 4.02 ± 0.21 | 4.36 ± 0.60 |
| MIP-1A | 1.75 ± 0.33 | 1.36 ± 0.28 | 2.25 ± 0.47 |
| IL-4 | 0.60 ± 0.12 | 0.50 ± 0.17 | 0.63 ± 0.17 |
| IL-1B | 0.45 ± 0.09 | 0.23 ± 0.07 | 0.33 ± 0.05 |
| IL-2 | 0.42 ± 0.09 | 0.22 ± 0.04 | 0.35 ± 0.08 |
| IL-6 | 6.37 ± 1.21 | 10.62 ± 4.66 | 6.98 ± 1.61 |
| EGF | 0.17 ± 0.06 | 0.14 ± 0.04 | 0.28 ± 0.12 |
| IL-13 | 0.60 ± 0.12 | 0.94 ± 0.44 | 0.51 ± 0.13 |
| IL-10 | 0.61 ± 0.12 | 0.35 ± 0.09 | 0.67 ± 0.17 |
| IL-12p70 | 0.65 ± 0.14 | 0.24 ± 0.04 | 0.60 ± 0.16 |
| IFNgamma | 0.76 ± 0.24 | 3.18 ± 1.52 | 0.86 ± 0.32 |
| IL-5 | 0.61 ± 0.17 | 0.47 ± 0.07 | 0.70 ± 0.10 |
| IL-17A | 0.30 ± 0.10 | 0.16 ± 0.06 | 0.21 ± 0.05 |
| IL-18 | 219.9 ± 77.8 | 26.22 ± 0.86 | 96.70 ± 20.08 |
| MCP-1 | 4.72 ± 0.68 | 10.81 ± 3.19 | 11.23 ± 3.95 |
| IP-10 | 1.63 ± 0.19 | 2.42 ± 0.29 | 2.67 ± 0.26 |
| GRO KC | 12.01 ± 0.71 | 8.81 ± 0.94 | 10.43 ± 1.83 |
| VEGF | 208.8 ± 38 | 98.2 ± 11 | 198.7 ± 50 |
| FRACTAL | 11.56 ± 2.25 | 2.68 ± 0.38 | 9.92 ± 3.11 |
| KINE | | | |
| LIX | 11.29 ± 2.85 | 8.41 ± 0.67 | 12.75 ± 2.23 |
| MIP-2 | 2.65 ± 0.32 | 1.43 ± 0.34 | 2.66 ± 0.70 |
| TNFalpha | 0.06 ± 0.02 | <0.02 | 0.07 ± 0.02 |
| RANTES | 0.20 ± 0.03 | 0.48 ± 0.12 | 0.28 ± 0.05 |

SEQUENCE LISTING

CD39L3 AA
SEQ ID No: 1
MVTVLTRQPCEQAGLKALYRTPTIIALVVLLVSIVVLVSITVIQIHKQEVLPPGLKYGIVLDAGSSRTTVYVYQWPAEKENNT
GVVSQTFKCSVKGSGISSYGNNPQDVPRAFEECMQKVKGQVPSHLHGSTPIHLGATAGMRLLRLQNETAANEVLESIQSYFKS
QPFDFRGAQIISGQEEGVYGWITANYLMGNFLEKNLWHMWVHPHGVETTGALDLGGASTQISFVAGEKMDLNTSDIMQVSLYG
YVYTLYTHSFQCYGRNEAEKKFLAMLLQNSPTKNHLTNPCYPRDYSISFTMGHVFDSLCTVDQRPESYNPNDVITFEGTGDPS
LCKEKVASIFDFKACHDQETCSFDGVYQPKIKGPFVAFAGFYYTASALNLSGSFSLDTFNSSTWNFCSQNWSQLPLLLPKFDE
VYARSYCFSANYIYHLFVNGYKFTEETWPQIHFEKEVGNSSIAWSLGYMLSLTNQIPAESPLIRLPIEPPVFVGTLAFFTAAA
LLCLAFLAYLCSATRRKRHSEHAFDHAVDSD.
CD39L3 NT
SEQ ID No: 2
ATGGTCACTGTGCTGACCCGCCAACCATGTGAGCAAGCAGGCCTCAAGGCCCTCTACCGAACTCCAACCATCATTGCCTTGGT
GGTCTTGCTTGTGAGTATTGTGGTACTTGTGAGTATCACTGTCATCCAGATCCACAAGCAAGAGGTCCTCCCTCCAGGACTGA
AGTATGGTATTGTGCTGGATGCCGGGTCTTCAAGAACCACAGTCTACGTGTATCAATGGCCAGCAGAAAAAGAGAATAATACC
GGAGTGGTCAGTCAAACCTTCAAATGTAGTGTGAAAGGCTCTGGAATCTCCAGCTATGGAAATAACCCCCAAGATGTCCCCAG
AGCCTTTGAGGAGTGTATGCAAAAAGTCAAGGGGCAGGTTCCATCCCACCTCCACGGATCCACCCCCATTCACCTGGGAGCCA
CGGCTGGGATGCGCTTGCTGAGGTTGCAAAATGAAACAGCAGCTAATGAAGTCCTTGAAAGCATCCAAAGCTACTTCAAGTCC
CAGCCCTTTGACTTTAGGGGTGCTCAAATCATTTCTGGGCAAGAAGAAGGGGTATATGGATGGATTACAGCCAACTATTTAAT
GGGAAATTTCCTGGAGAAGAACCTGTGGCACATGTGGGTGCACCCGCATGGAGTGGAAACCACGGGTGCCCTGGACTTAGGTG
GTGCCTCCACCCAAATATCCTTCGTGGCAGGAGAGAAGATGGATCTGAACACCAGCGACATCATGCAGGTGTCCCTGTATGGC
TACGTATACACGCTCTACACACACAGCTTCCAGTGCTATGGCCGGAATGAGGCTGAGAAGAAGTTTCTGGCAATGCTCCTGCA
GAATTCTCCTACCAAAAACCATCTCACCAATCCCTGTTACCCTCGGGATTATAGCATCAGCTTCACCATGGGCCATGTATTTG
ATAGCCTGTGCACTGTGGACCAGAGGCCAGAAAGTTATAACCCCAATGATGTCATCACTTTTGAAGGAACTGGGGACCCATCT
CTGTGTAAGGAGAAGGTGGCTTCCATATTTGACTTCAAAGCTTGCCATGATCAAGAAACCTGTTCTTTTGATGGGGTTTATCA
GCCAAAGATTAAAGGGCCATTTGTGGCTTTTGCAGGATTCTACTACACAGCCAGTGCTTTAAATCTTTCAGGTAGCTTTTCCC
TGGACACCTTCAACTCCAGCACCTGGAATTTCTGCTCACAGAATTGGAGTCAGCTCCCACTGCTGCTCCCCAAATTTGATGAG
GTATATGCCCGCTCTTACTGCTTCTCAGCCAACTACATCTACCACTTGTTTGTGAACGGTTACAAATTCACAGAGGAGACTTG
GCCCCAAATACACTTTGAAAAAGAAGTGGGGAATAGCAGCATAGCCTGGTCTCTTGGCTACATGCTCAGCCTGACCAACCAGA
TCCCAGCTGAAAGCCCTCTGATCCGTCTGCCCATAGAACCACCTGTCTTTGTGGGCACCCTCGCTTTCTTCACAGCGGCAGCC
TTGCTGTGTCTGGCATTTCTTGCATACCTGTGTTCAGCAACCAGAAGAAAGAGGCACTCCGAGCATGCCTTTGACCATGCAGT
GGATTCTGACTGA
sol CD39L3 R67G T69R AA
SEQ ID No: 3
34567895123456789612345678971289
MQIHKQEVLPPGLKYGIVLDAGSS [symbol] VYVYQWPAEKENNTGVVSQTFKCSVKGSGISSYGNNPQDVPRAFEECMQKVKGQV
PSHLHGSTPIHLGATAGMRLLRLQNETAANEVLESIQSYFKSQPFDFRGAQIISGQEEGVYGWITANYLMGNFLEKNLWHMWV
HPHGVETTGALDLGGASTQISFVAGEKMDLNTSDIMQVSLYGYVYTLYTHSFQCYGRNEAEKKFLAMLLQNSPTKNHLTNPCY
PRDYSISFTMGHVFDSLCTVDQRPESYNPNDVITFEGTGDPSLCKEKVASIFDFKACHDQETCSFDGVYQPKIKGPFVAFAGF
YYTASALNLSGSFSLDTFNSSTWNFCSQNWSQLPLLLPKFDEVYARSYCFSANYIYHLFVNGYKFTEETWPQIHFEKEVGNSS
IAWSLGYMLSLTNQIPAESPLIRLPIEPPV.

1. A method of treatment comprising administering an apyrase agent to a subject at risk of or with fibroproliferative disorder.

2. The method of claim 1 wherein the fibroproliferative disorder is pulmonary hypertension.

3. The method of claim 2 wherein the pulmonary hypertension is pulmonary arterial hypertension.

4-6. (canceled)

7. The method of claim 1 wherein the fibroproliferative disorder is an acute fibroproliferative disorder.

8. The method of claim 7 wherein the acute fibroproliferative disorder develops after an injury selected from the group consisting of ischemic illness, environmental pollutants, alcohol poisoning, toxin exposure, acute respiratory distress syndrome, radiation, and chemotherapy.

9. The method of claim 7 wherein the acute fibroproliferative disorder is scleroderma, kidney fibrosis, cardiac fibrosis, pulmonary fibrosis, oral fibrosis, endomyocardial fibrosis, deltoid fibrosis, pancreatitis, inflammatory bowel disease, Grahn's disease, nodular fascilitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, glioblastoma in Li-Fraumeni syndrome, sporadic glioblastoma, myleoid leukemia, acute myelogenous leukemia, myelodysplastic syndrome, myeloproliferative syndrome, gynecological cancer, Kaposi's sarcoma, Hansen's disease, collagenous colitis, acute fibrosis, systemic sclerosis, and fibrosis arising from tissue or organ transplant or graft rejection.

10. The method of claim 1 wherein the fibroproliferative disorder is interstitial fibrosis.

11. The method of claim 10 wherein the interstitial fibrosis is renal interstitial fibrosis or interstitial pulmonary fibrosis.

12. The method of claim 1 wherein the fibroproliferative disorder is posthrombotic syndrome associated with venous thrombosis.

13. The method of claim 1 wherein the fibroproliferative disorder is associated with an increased risk of, or occurrence of, ventricular heart failure.

14. (canceled)

15. The method of claim 3 wherein the pulmonary arterial hypertension is associated with an increased risk of, or occurrence of, ventricular heart failure.

16. The method of claim 12 wherein the posthrombotic syndrome associated with venous thrombosis is further associated with an increased risk of, or occurrence of, ventricular heart failure.

17-20. (canceled)

21. The method of claim 1 wherein the apyrase agent is administered about 2 to about 50 times, optionally about to about 24 times, or optionally about 4 to about 24 times.

22. (canceled)

23. The method of claim 21 wherein the apyrase agent is a soluble apyrase agent

24. The method of claim 21 wherein the apyrase agent is homologous to SEQ ID No: 1.

25. The method of claim 21 wherein the apyrase agent is a recombinant apyrase agent produced in CHO cells.

26. The method of claim 21 wherein the subject is further administered one or more of antiplatelets, anticoagulants, and thrombolytics.

27. The method of claim 1 wherein the subject, after the apyrase agent administering step, achieves one or more of a sustained PAP reduction, reversal of vascular remodeling and fibrosis, and improvement in RV function.

28. (canceled)

29. A method of treatment of a subject comprising quantifying the concentrations of one or more cytokines of Table 3 in plasma of the subject and in individuals with no known pathologies, wherein if said subject has an elevation in the concentration of one or more of said cytokines relative to the individuals with no known pathologies, one or more agents useful for treating pulmonary arterial hypertension is administrated.

30. A method of treatment of a subject comprising quantifying the concentrations of one or more cytokines of Table 1 in BALF of the subject and in individuals with no known pathologies, wherein if said subject has a decrease in the concentration of one or more of said cytokines relative to the individuals with no known pathologies, one or more agents useful for treating pulmonary arterial hypertension is administrated.

31-32. (canceled)


SEQUENCE LISTING (3 sequences)

SEQ ID NO: 1 - protein, 529 aa, Homo sapiens
  misc_feature (1)...(529): CD39L3
MVTVLTRQPCEQAGLKALYRTPTIIALVVLLVSIVVLVSITVIQIHKQEVLPPGLKYGIVLDAGSSRTTVYVYQWPAEKENNTGVVSQTFKCSVKGSGISSYGNNPQDVPRAFEECMQKVKGQVPSHLHGSTPIHLGATAGMRLLRLQNETAANEVLESIQSYFKSQPFDFRGAQIISGQEEGVYGWITANYLMGNFLEKNLWHMWVHPHGVETTGALDLGGASTQISFVAGEKMDLNTSDIMQVSLYGYVYTLYTHSFQCYGRNEAEKKFLAMLLQNSPTKNHLTNPCYPRDYSISFTMGHVFDSLCTVDQRPESYNPNDVITFEGTGDPSLCKEKVASIFDFKACHDQETCSFDGVYQPKIKGPFVAFAGFYYTASALNLSGSFSLDTFNSSTWNFCSQNWSQLPLLLPKFDEVYARSYCFSANYIYHLFVNGYKFTEETWPQIHFEKEVGNSSIAWSLGYMLSLTNQIPAESPLIRLPIEPPVFVGTLAFFTAAALLCLAFLAYLCSATRRKRHSEHAFDHAVDSD

SEQ ID NO: 2 - DNA, 1590 bp, Homo sapiens
  misc_feature (1)...(1590): CD39L3
atggtcactgtgctgacccgccaaccatgtgagcaagcaggcctcaaggccctctaccgaactccaaccatcattgccttggtggtcttgcttgtgagtattgtggtacttgtgagtatcactgtcatccagatccacaagcaagaggtcctccctccaggactgaagtatggtattgtgctggatgccgggtcttcaagaaccacagtctacgtgtatcaatggccagcagaaaaagagaataataccggagtggtcagtcaaaccttcaaatgtagtgtgaaaggctctggaatctccagctatggaaataacccccaagatgtccccagagcctttgaggagtgtatgcaaaaagtcaaggggcaggttccatcccacctccacggatccacccccattcacctgggagccacggctgggatgcgcttgctgaggttgcaaaatgaaacagcagctaatgaagtccttgaaagcatccaaagctacttcaagtcccagccctttgactttaggggtgctcaaatcatttctgggcaagaagaaggggtatatggatggattacagccaactatttaatgggaaatttcctggagaagaacctgtggcacatgtgggtgcacccgcatggagtggaaaccacgggtgccctggacttaggtggtgcctccacccaaatatccttcgtggcaggagagaagatggatctgaacaccagcgacatcatgcaggtgtccctgtatggctacgtatacacgctctacacacacagcttccagtgctatggccggaatgaggctgagaagaagtttctggcaatgctcctgcagaattctcctaccaaaaaccatctcaccaatccctgttaccctcgggattatagcatcagcttcaccatgggccatgtatttgatagcctgtgcactgtggaccagaggccagaaagttataaccccaatgatgtcatcacttttgaaggaactggggacccatctctgtgtaaggagaaggtggcttccatatttgacttcaaagcttgccatgatcaagaaacctgttcttttgatggggtttatcagccaaagattaaagggccatttgtggcttttgcaggattctactacacagccagtgctttaaatctttcaggtagcttttccctggacaccttcaactccagcacctggaatttctgctcacagaattggagtcagctcccactgctgctccccaaatttgatgaggtatatgcccgctcttactgcttctcagccaactacatctaccacttgtttgtgaacggttacaaattcacagaggagacttggccccaaatacactttgaaaaagaagtggggaatagcagcatagcctggtctcttggctacatgctcagcctgaccaaccagatcccagctgaaagccctctgatccgtctgcccatagaaccacctgtctttgtgggcaccctcgctttcttcacagcggcagccttgctgtgtctggcatttcttgcatacctgtgttcagcaaccagaagaaagaggcactccgagcatgcctttgaccatgcagtggattctgactga

SEQ ID NO: 3 - protein, 444 aa, Homo sapiens
  misc_feature (1)...(444): R67G T69R mutant of soluble CD39L3
MQIHKQEVLPPGLKYGIVLDAGSSGTRVYVYQWPAEKENNTGVVSQTFKCSVKGSGISSYGNNPQDVPRAFEECMQKVKGQVPSHLHGSTPIHLGATAGMRLLRLQNETAANEVLESIQSYFKSQPFDFRGAQIISGQEEGVYGWITANYLMGNFLEKNLWHMWVHPHGVETTGALDLGGASTQISFVAGEKMDLNTSDIMQVSLYGYVYTLYTHSFQCYGRNEAEKKFLAMLLQNSPTKNHLTNPCYPRDYSISFTMGHVFDSLCTVDQRPESYNPNDVITFEGTGDPSLCKEKVASIFDFKACHDQETCSFDGVYQPKIKGPFVAFAGFYYTASALNLSGSFSLDTFNSSTWNFCSQNWSQLPLLLPKFDEVYARSYCFSANYIYHLFVNGYKFTEETWPQIHFEKEVGNSSIAWSLGYMLSLTNQIPAESPLIRLPIEPPV